(19) United States
(12) Patent Application Publication
(10) Pub. No.: US 2007/0250051 A1
(43) Pub. Date: Oct. 25, 2007

(54) Heating via microwave and millimeter-wave transmission using a hypodermic needle

(76) Inventors: Kerry R. Gaston, Montgomery, AL (US); Harold W. Cox, Montgomery, AL (US); Victor L. Granatstein, Silver Spring, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(21) Appl. No.: 11/411,701

(22) Filed: Apr. 25, 2006

(51) Int. Cl. A61B 18/18; A61F 2/00

(52) U.S. Cl. 606/33; 607/100

(57) ABSTRACT

A system and method for treating biological tissues with EM energy in the millimeter or microwave range utilizes a needle to position a source of EM radiation proximate the tissue to be treated. An absorption aid such as sterile water or saline may be used in an embodiment of the invention to aid in absorption of the incident EM energy in the area of interest. In an embodiment of the invention, the EM energy is tuned in wavelength and power to effect optimum treatment with minimal damage to surrounding tissues.

### FIELD OF THE INVENTION

The invention pertains to the heating of biological tissue, and pertains more particularly in certain embodiments of the invention to the heating of human or animal tissue via microwave radiation.

### BACKGROUND OF THE INVENTION

Electromagnetic radiation has been shown to have beneficial effects on living tissue in a number of application. For example, known techniques utilize electromagnetic radiation to destroy or inhibit interior tumors by heating and thereby destroying tumor cells. However, the usefulness of employing electromagnetic radiation is limited by the medical practitioner's ability to focus the electromagnetic radiation into the volume of interest while achieving sufficient penetration of a substantial portion of the radiation at the required depth.

For example, known techniques radiate electromagnetic energy via an antenna which can be inserted into the tumor. However, such techniques, using radiation in the range of about 400 MHz to 6 GHz, demonstrate no known ability to vary the depth of penetration of the radiation itself as opposed to the antenna. During tumor destruction, over-penetration of the radiation can damage surrounding healthy tissues, while under-penetration can leave some or all of the tumor untreated. Moreover, existing techniques largely lack the ability to increase the local absorption of the delivered radiation independent of the absorption characteristics of the biological tissue in question, contributing to the likelihood of under treatment or over treatment. For example, under treatment can result when the applied power is reduced to preserve surrounding healthy tissues which would be otherwise damaged by the required dose given the absorption characteristics of the tissues in question. Likewise, over treatment can occur when the full dose required to treat the tumor is used and the absorption characteristics of the tissue in question allow over penetration.

### BRIEF SUMMARY OF THE INVENTION

Embodiments of the invention provide a mechanism for heating of a biological tissue such as a tumor or diseased region via microwave and other electromagnetic radiation for therapeutic purposes. The exposure of the target tissue to EM energy of the proper wavelength and power for the proper duration result in destruction of the tissue. It is important however to minimize destruction of surrounding healthy or non-targeted tissues. Embodiments of the invention provide a mechanism for controlling the placement and penetration of applied microwave radiation such that the targeted cells may be fully treated while avoiding significant damage to other tissue. Embodiments of the invention employ a needle for applying a microwave field to an internal target volume while also using power and frequency tuning and/or water content control to adjust the depth of penetration of the applied radiation to minimize penetration beyond the target volume.

The needle may also contain one or more sensors to locate and/or view the treated area, and/or to observe the effect of treatment. Suitable sensors include light pipes or other imaging sensors as well as thermal sensors.

In an embodiment of the invention, a coaxial cable is used within the needle to deliver the EM energy. In other embodiments of the invention, other arrangements may be employed instead. In one embodiment of the invention, the needle itself may serve as a hollow waveguide. In alternative embodiment of the invention, the needle serves as the outer conductor of a coaxial conductor arrangement. The inner wall of the needle is preferably coated with a highly conductive material such as gold in embodiments of the invention wherein the electrical properties of the needle itself are exploited to deliver EM energy.

In an embodiment of the invention, an absorption aid such as sterile water or saline solution is injected into the target area. This has the effect of increasing the absorption of EM energy in the target area, improving treatment and preventing over-penetration into surrounding healthy tissues.

### BRIEF DESCRIPTION OF THE SEVERAL VIEWS OF THE DRAWINGS

FIG. 1 is a schematic view of a patient and a treatment system according to an embodiment of the invention, showing the typical use of the referenced embodiment;

FIG. 2 is a more particularized schematic view of the system according to an embodiment of the invention showing the needle, as well as the EM energy delivery mechanism and source;

FIG. 3 is a power absorption plot showing the residual power as a function of distance for different starting powers;

FIG. 4 is a power absorption plot showing the residual power as a function of distance for different EM wavelengths of the same initial power; and

FIG. 5 is a flow chart showing a process of treatment according to an embodiment of the invention.

### DETAILED DESCRIPTION OF THE INVENTION

The following description illustrates certain embodiments of the invention in the best mode of practice currently known, but should not be construed as in any way limiting the scope of the described embodiments or any other embodiments of the invention. Rather, the invention is limited only by the attached claims, which are purposefully drafted to cover the claimed aspects of the invention and all equivalents without being limited to the specific examples described herein.

The described embodiments of the invention provide a way to cause controlled heating of a biological tissue such as a tumor or diseased region via microwave and other electromagnetic radiation for therapeutic purposes. Microwaves will be used as an example herein, but it will be appreciated that the this term can also include millimeter waves and other wavelengths that fall outside of a strictly technical definition of “microwave.”

Generally, microwaves heat exposed substances by causing water molecules with the substance to rapidly vibrate. Biological cells, whether human or other animal, are comprised primarily of water, such that in general, the human or animal body itself is comprised largely of water. For example, the adult human body comprises about 50 to 70 percent water, and a child's body may comprise as much as 75 percent water or more. With respect to specific tissue types, the cells of the human brain comprise about 70 percent water, the cells of human lungs comprise about 90 percent water, and the cells of the human blood comprise about 83 percent water. For treating a cancerous tumor or other unwanted growth or tissue, the goal of microwave heating is to heat the constituent cells of the tissue to the point that they rupture or are otherwise rendered inactive.

However, since many targeted biological tissues (e.g., cancerous tissue) have microwave absorption characteristics that are similar or identical to those of other surrounding tissues (e.g., healthy tissue), it is difficult to limit the effect of microwave heating to the targeted tissue. This unavoidable destruction of healthy tissue is undesirable and may also cause health problems for the patient. Moreover, if the radiation is reduced in dosage or power to avoid harm to healthy tissues, the unfortunate result is often that the targeted tissues are also left at least partially intact. In the case of living biological tissue such as cancer however, a seed of leftover targeted material may lead to a growth and recurrence of the problem. This is still a problem, although not as significant, when the targeted cells are not capable of sustained independent growth. Fat cells are examples of this type of tissue.

Embodiments of the invention combine several improvements to provide a mechanism for controlling the placement and penetration of applied microwave radiation such that targeted cells may be fully treated while avoiding significant damage to surrounding healthy (or non-targeted) tissue. In overview, embodiments of the invention employ a needle for applying a microwave field to an internal target volume while also using power and frequency tuning and/or water content control to adjust the depth of penetration of the applied radiation to minimize penetration beyond the target volume.

We have noted that the absorption and penetration of microwave radiation in biological tissues is wavelength dependent. In other words, longer wavelengths (i.e., lower energies) tend to penetrate further than lower wavelengths (i.e., higher energies). Thus, wavelength tuning can be used to reach either very small volumes of target tissue at the surface of the patient or larger volumes of target tissue deeper in the patient. However, wavelength tuning alone will not always allow the precise targeting of a small target volume that is located deeper in the patient, because the large depth of penetration needed to reach the target is sometimes incompatible with the microwave energy being concentrated in a small volume.

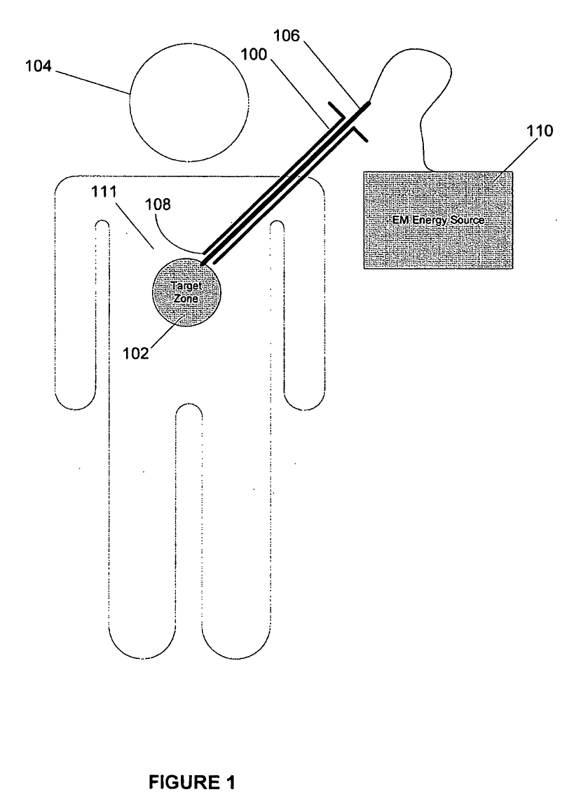

In an embodiment of the invention, to overcome the aforementioned problem, a needle 100 (shown larger than scale for ease of understanding) is used to reach the target area 102 (shown larger than is typical for ease of understanding) within the patient 104. In particular, as illustrated in FIG. 1, a hollow needle 100 containing a coaxial cable 106 is inserted into the patient and reaches approximately to or into the target area 102. It will be appreciated that the items shown in FIG. 1, and in particular the needle width dimensions, are not shown to scale for the sake of clarity. In an embodiment of the invention, the needle 100 is a hypodermic needle of approximately 0.04 inches in interior diameter, approximately 0.005 inches wall thickness, and an outer or exterior diameter of approximately 0.05 inches. The needle is preferably adapted depending upon the depth within the body at which the tumor resides.

The coaxial cable 106 should be thin enough to fit within the needle 100 and to be able to exit the opening 108 in the tip of the needle 100. Commercially available coaxial cable of 0.864 mm diameter is suitable in certain embodiments of the invention, but other sizes may be alternatively used within the constraints outlined above. In operation, the needle 100 is inserted automatically or by a medical practitioner such as a nurse, doctor, or surgeon, into the patient 104 and up to or into the target area 102. The positioning of the needle may be by any traditional broad view technique such as x-ray, ultrasound, etc., or may be based on calculations given the known location of the target area.

At that point, the core of the coaxial cable 106 is extended out the tip 108 of the needle 100 such that it is substantially proximate to the target area 102, i.e. within, touching, or very close to the target area 102. The tip of the cable 106 serves as a monopole antenna. Once the coaxial cable 106 is positioned, a microwave source 110 attached to the coaxial cable transmits microwave energy into the coaxial cable 106, for emission from the tip of the cable 106 into the target area 102. In general, the tip of the inner conductor of the co-axial cable will extend beyond the end of the outer conductor. The distance between the tip of the inner conductor and the end of the outer conductor, and the distance between the end of the outer conductor and the tip of the needle will each be chosen to optimize the antenna radiation pattern. Such an antenna has been shown to generate a local ‘hot spot’ in its near field region just beyond the tip of the coaxial inner conductor. To concentrate the microwave energy in the target volume, the end of the coaxial cable will be situated so that the location of the ‘hot spot’ coincides with the location of the target zone.

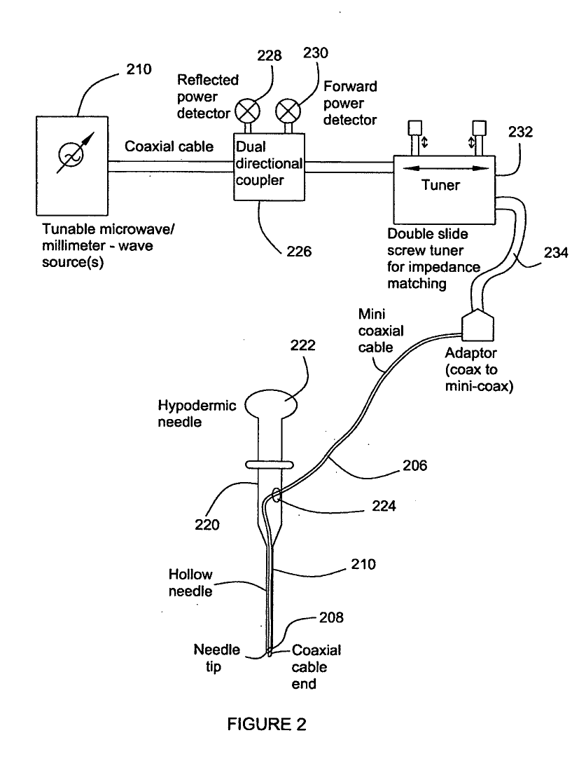

The microwave source 110, needle 100 and coaxial cable 106 and their interactions are discussed further with respect to FIG. 2. These elements are illustrated in FIG. 2 via elements 210, 200, and 206 respectively. As shown in FIG. 2, the needle 210 is a hollow needle which, in an embodiment of the invention, is connected to a larger hollow shaft 220. The larger hollow shaft 220 may have a handle 222 attached thereto for easy manipulation of the needle 210. The handle may also be a flexible hollow bulb to allow the delivery of fluid through the needle 210 to the target site in an embodiment of the invention. This embodiment will be discussed in greater detail later herein.

In addition, in an embodiment of the invention, the hollow shaft 220 contains an opening 224 to allow for the introduction of the coaxial cable 206 into the shaft 220 and needle 210. Although the tip of the coaxial cable 206 is shown slightly extending from the tip 208 of the needle 210 as it would be in an embodiment of the invention during radiation delivery, it will be appreciated that the tip of the cable 106 may be retracted or extended as needed during operation of the device.

The coaxial cable 206 is shown connected to the microwave source 210 via a series of intermediate devices. In particular, the microwave source 210 is connected to a dual directional coupler 226 in an embodiment of the invention. The microwave source 210 and dual directional coupler 226 may be connected via any suitable mechanism such as a coaxial cable. The dual directional coupler 226 further comprises a reflected power detector 228 and a forward power detector 230. The reflected power detector 228 measures reflected energy to aid a tuner in effectively coupling to the remainder of the system. The dual directional coupler 226 is in turn coupled to a tuner 232 such as a double slide screw tuner for impedance matching in an embodiment of the invention. The tuner is used in an embodiment of the invention to minimize reflected energy as frequency is varied. This enables efficient transmission of the microwave signal into a coaxial cable 234. The coaxial cable 234 may be an ordinary coaxial cable. Finally, if the coaxial cable 234 is an ordinary cable rather than a miniature cable, then in an embodiment of the invention, an adapter 236 is used to couple cable 234 to cable 206. The cables 206 are 234 are preferably 50 ohm cables such as RG-8 or RG-58. However, any other cable type including but not limited to RG-6, RG-11 and RG-59 of any other impedance may also be used.

The co-axial components shown in FIG. 2 are generally commercially available at frequencies up to 40 GHz from a number of suppliers such as ADVANCED TECHNICAL MATERIALS, INC. of Patchogue, N.Y. For higher frequencies, it is preferable to use equivalent waveguide components which are generally available but are more costly.

In an embodiment of the invention, the dual directional coupler 226 allows for sampling a fixed small percentage of the power flow in each direction and the detectors convert the microwaves to easily readable DC signals. In a further embodiment of the invention, the double-slide screw tuner 232 for impedance matching allows one to maximize power absorbed in the tumor and to minimize reflected power. The effectiveness of the adjustments can be measured with the aid of the dual directional coupler 226 and detectors previously described.

The microwave source 210 may be of any suitable configuration. In an embodiment of the invention, an IMPATT diode oscillator such as can be obtained from QUINSTAR TECHNOLOGY, INC. of Torrance, Calif., having a power output of approximately 1 Watt cw. If higher power and/or greater tunability are needed or desired, a Traveling Wave Tube (TWT) amplifier driven by a Gunn diode oscillator is used in an embodiment of the invention. One supplier of TWT amplifiers is HUGHES ELECTRONICS of Germantown, Md., and QUINSTAR also supplies tunable Gunn oscillators. There are a number of other possible sources that will be known to those of skill in the art depending on desired power, frequency range and tunability.

In a further embodiment of the invention, the needle 208 also contains one or more sensors. One type of sensor usable within this embodiment of the invention is a light pipe or other mechanism for viewing the treated area, and may be used to help locate the needle 210 within the patient and/or to observe the effect of treatment. Another type of sensor that may additionally or alternatively be used is a thermal sensor for detecting the temperature rise in the treated area.

Although the system is described above according to an embodiment wherein a needle and contained coaxial cable are used, other arrangements may be employed instead without departing from the scope of the invention. For example, the needle 210 itself may serve as a hollow waveguide, making unnecessary the use of an internal cable. Alternatively, the needle 210 may serve as the outer conductor of a coaxial conductor arrangement. In this case, an insulating dielectric material is preferably used to isolate the needle from the inner conductor. In addition, whether the needle serves as a conductor or waveguide, it is desirable in an embodiment of the invention to coat the inner wall of the needle 210 with a highly conductive material such as gold.

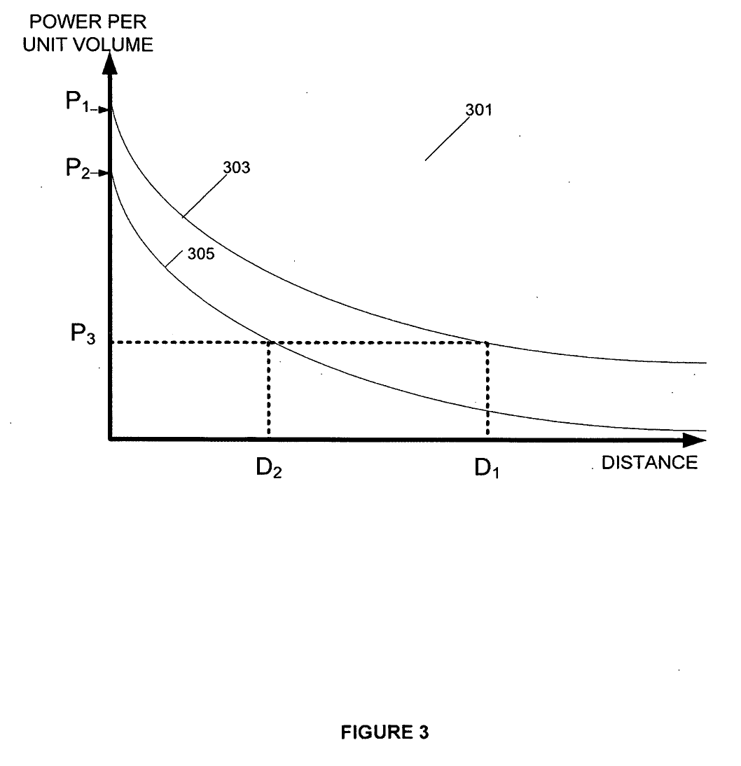

The system described above enables the practitioner to selectively dose the target area with microwave energy, but the parameters of that energy are also significant in an embodiment of the invention. In particular, both the wavelength and power of the delivered radiation are selectable in an embodiment of the invention to maximize radiation absorption in the target tissue and to minimize radiation absorption in the surrounding healthy tissue 111 (see FIG. 1). To aid in understanding the effect of wavelength and power, FIG. 3 illustrates the generalized notional power absorption characteristics of radiated tissue.

In particular, the graph 301 of FIG. 3 shows two power curves, 303, 305, representing the absorption characteristics of the tissue at two different power settings. The higher curve 303 represents a power per unit volume P1 at the center of the near field ‘hot spot’, whereas the lower curve 305 represents a lower power per unit volume of P2 at the center of the ‘hot spot’. As can be seen the shapes of the curves, 303, 305, are similar but the power per unit volume of the higher curve 303 stays generally higher than that of the lower curve 305 due to a higher power being supplied by the antenna. Assuming a threshhold power per unit volume of P3 to adequately treat the target tissue, it can be seen that power per unit volume at the center of the ‘hot spot’ P1 provides treatment to a distance of D1 whereas power per unit volume P2 provides treatment only out to a lesser distance D2.

The shapes of the power curves are due to both absorption and the near field antenna pattern. In other words, even with no absorption at all, the power curve will still fall off as distance from the center of the ‘hot spot’ increases. The absorption of power by the tissue tends to increase the rate of fall off, i.e., to steepen the power curve, which is especially noticeable at short distances. In addition, the wavelength of the applied radiation affects the rate of absorption, thus also affecting the steepness of the power curves. It has been noted that longer wavelengths exhibit a lower rate of absorption, while shorter wavelengths exhibit a higher rate of absorption.

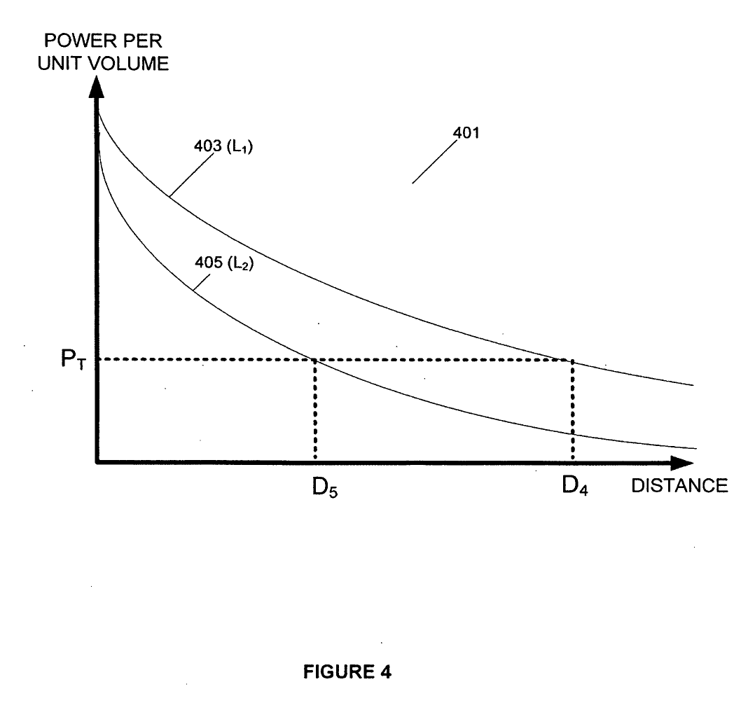

FIG. 4 illustrates the impact of wavelength on the absorption curves. In particular, the graph 401 of FIG. 4 illustrates two generalized notional curves 403, 405. The first curve 403 shows the power absorption characteristics of biological tissue at a first wavelength L₁, whereas the second curve shows the power absorption characteristics of biological tissue at a second wavelength L₂, with L₂ being smaller than L₁. As can be seen, the absorption of the radiation at L₁ is less (and thus its penetration is greater) due to its longer wavelength. Thus, using the shorter wavelength L2 provides the advantages of less heating of surrounding healthy tissue and of minimizing the energy required to heat and destroy the tumor. We note specifically that depth of penetration in biological tissue varies from 3 mm down to 0.4 mm as RF frequency rises from 10 GHz to 100 GHz, which range encompasses both microwaves and millimeter waves (for frequency above 30 GHz). Thus, still referring to FIG. 4, given a treatment power threshold of P_{T}, it can be seen that EM energy of wavelength L₁ will affect tissues out to distance D₄, whereas energy of shorter wavelength L₂ will affect tissues only out to a lesser distance D₅.

Thus, as can be seen from the foregoing discussion, the spatial impact of applied radiation depends upon the applied power and the applied wavelength. As such, the power and wavelength of the incident radiation used for treatment are modified in an embodiment of the invention to tune the penetration of the radiation such that the target tissue is effectively treated while minimizing damage to surrounding healthy tissue.

As noted above, the absorption characteristics of biological tissue are wavelength dependent, and that dependence is exploited in an embodiment of the invention. However, it is also desirable to alter the makeup of the biological tissue itself to provide further adjustment with respect to the absorption characteristics and depth of penetration. To this end, in an embodiment of the invention, an absorptive material such as water or other liquid is injected into the target volume to increase the absorption of EM energy in that volume. This has the dual related benefits of increasing the destruction of unwanted cells in the target zone while limiting over penetration of the EM energy into surrounding healthy tissues.

In an embodiment of the invention, the injection of the absorptive material into the target volume is performed via the same needle used to deliver the antenna to the area. In this embodiment of the invention, referring to FIG. 2, the fluid may be delivered through the needle 210 around the coaxial cable 206 while the cable 206 is in place, or alternatively, may be delivered prior to insertion of the cable 206 into the needle 210. The handle 222 in this embodiment of the invention may comprise a hollow compressible bulb for containing the fluid and for being squeezed to eject the fluid into needle 210 and into the target area. Alternatively, a different attached pump mechanism or a separate pump mechanism may be used.

In an alternative embodiment of the invention, a different needle is used to deliver the absorptive fluid. In this embodiment of the invention, the fluid may be injected into one or more locations within the target volume prior to insertion of the RF treatment needle 210. For example, with respect to tissues with low diffusion characteristics, multiple injections into different areas of the target volume are desirable at the discretion of the treating practitioner.

Although the foregoing embodiment of the invention has been described as employing water as the absorptive fluid to be injected, it will be appreciated that any fluid having increased millimeter wave or microwave absorption may be used. However, it is preferable that the injected fluid is additionally sterile, hypoallergenic, and nontoxic, to a degree sufficient to avoid causing an adverse reaction to the fluid in the patient.

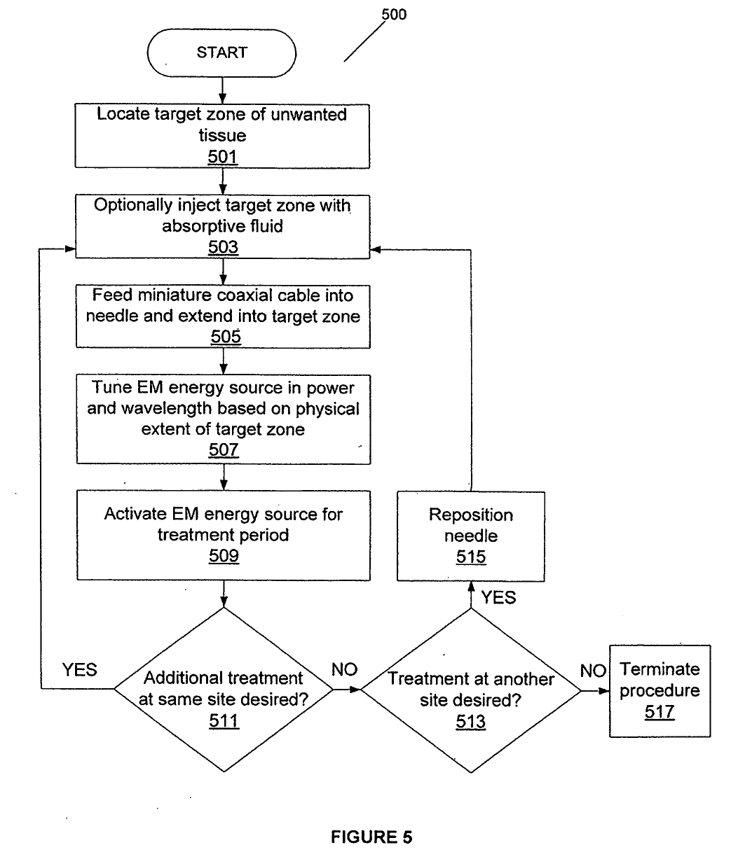

Having described a number of improvements corresponding to certain embodiments of the invention, the integrated use of these techniques according to an embodiment of the invention will be described with reference to the flow chart of FIG. 5. In particular, the flow chart 500 illustrates one treatment course wherein an identified unwanted tissue mass is treated via exposure to millimeter or microwave EM energy. Although the process described with respect to the flow chart 500 will focus by way of example on a construction that employs a miniature coaxial cable within the needle and wherein an absorptive fluid is injected via the needle prior to treatment, it will be appreciated that the process according to other embodiments of the invention includes the use of the other alternative techniques and structures described above in the manner described in FIG. 5 where appropriate.

At step 501, a target zone of unwanted tissue is located. The target zone has a physical extent, i.e., a linear dimension such as radius or diameter, and a location. The target zone may be identified via any traditional means such as x-ray, ultrasound, touch, etc. If needed, the target zone is optionally injected with an absorptive fluid such as sterile water or saline in step 503 to improve its absorptivity. The injection may be accomplished via the needle used for radiation or via a separate needle. In this example, it will be assumed that the injection is accomplished via the same needle to be used for radiation. Thus, after the fluid injection, the needle is left in place and the miniature coaxial cable is fed into the needle and extended into a proximate relationship with the target zone in step 505.

At step 507, a microwave/millimeter wave source connected to the coaxial cable is tuned in power and wavelength based on the physical extent of the target zone to assure effective treatment within the target zone while minimizing heating in surrounding healthy tissue. The source is activated at step 509 for a treatment period, thus exposing the target zone to microwave/millimeter EM energy during this period. The length of the treatment period may be predetermined based on empirical indications or may be variable based on feedback during the process as discussed above.

At step 511, it is determined whether an additional treatment at the same site is desired. For example, in addition to heating through EM absorption, there may also be heating via thermal conduction within the target zone as well as from the target zone to the surrounding tissues. For this reason, it is desirable in an embodiment of the invention to perform a series of short treatments at the same site. If it is determined that an additional treatment at the same site is desired, the process returns to step 503 (or 505 if additional fluid is not needed) and continues from there.

Otherwise the process continues to step 513 wherein it is determined whether an additional treatment at another site within the target zone or at another target zone is required. For example, an elongated or asymmetric zone of unwanted tissue may be viewed as a series or collection of cubical or spherical target zones, so that a series of treatments, one or more in each constituent zone, may be needed to treat the entire area of unwanted tissue. If at step 513 it determined that an additional treatment at another site within the target zone or at another target zone is required, the needle is repositioned in step 515 and the process continues at step 503. Otherwise, the process terminates at step 517.

It will be appreciated that certain embodiments of the invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

Although the method is described primarily with respect to a tumor, it will be appreciated that any tissue or material that is susceptible to treatment via millimeter or microwave radiation can benefit from the invention. Although such material will generally be biological, such is not a requirement. Moreover, it will be appreciated that with respect to living entities, the invention is equally applicable to humans and animals alike.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

1. A method for treating a target zone of biological tissue within a subject with electromagnetic radiation, the electromagnetic radiation having a power and wavelength and the target zone having a location and a physical extent, the system comprising:
determining the location and physical extent of the target zone;
based on the determined location of the target zone, inserting a hollow needle into the subject such that the tip of the needle is proximate the target zone, wherein the hollow needle comprises an inner passage and wherein a conductor is located within the inner passage;
determining a wavelength of electromagnetic radiation to be used to treat the biological tissue of the target zone based on the physical extent of the target zone; and
applying a signal to the conductor within the inner passage of the hollow needle to cause the emission of electromagnetic radiation having the determined a wavelength from the conductor, whereby the emitted electromagnetic radiation contacts the biological tissue of the target zone.

2. The method according to claim 1, wherein the step of determining a wavelength of electromagnetic radiation to be used to treat the biological tissue of the target zone further comprises determining a power of electromagnetic radiation.

3. The method according to claim 1, further comprising the step of injecting an fluid into the target zone, wherein the fluid is absorptive at the determined wavelength.

4. The method according to claim 3, wherein the fluid is injected into the target zone via the hollow needle.

5. The method according to claim 3, wherein the fluid is injected into the target zone via a passage separate from the hollow needle.

6. The method according to claim 1, further comprising the step extending the conductor out of the inner passage of the hollow needle and into the target zone.

7. The method according to claim 1, wherein the step of applying a signal to the conductor within the inner passage of the hollow needle comprises applying the signal for a predetermined period of time.

8. The method according to claim 1, wherein the step of applying a signal to the conductor within the inner passage of the hollow needle comprises applying the signal for a period of time determined by a reaction of the biological tissue to the emitted electromagnetic radiation.

9. The method according to claim 1, wherein the step of applying a signal to the conductor within the inner passage of the hollow needle comprises applying the signal for a period of time determined by a reaction of the biological tissue to the emitted electromagnetic radiation.

10. The method according to claim 1, wherein the inner conductor comprises a coaxial cable.

11. The method according to claim 1, wherein the inner conductor and the hollow needle together comprise a coaxial cable, and wherein the inner conductor and the hollow needle are held in a spaced apart relationship by an insulating material.
